(19) United States
(12) Patent Application Publication
(10) Pub. No.: US 2025/0176866 A1
(43) Pub. Date: Jun. 5, 2025

(54) IMPLANTABLE BIOSENSORS

(71) Applicant: The Texas A&M University System, College Station, TX (US)

(72) Inventors: Michael J. McShane, College Station, TX (US); Aniket Biswas, College Station, TX (US); Ashvin Nagaraja, Bryan, TX (US); Gerard L. Cote, College Station, TX (US); Michael V. Pishko, College Station, TX (US)

(73) Assignee: The Texas A&M University System, College Station, TX (US)

(21) Appl. No.: 19/035,737

(22) Filed: Jan. 23, 2025

Related U.S. Application Data
(63) Continuation of application No. 16/060,264, filed on Jun. 7, 2018.
(60) Provisional application No. 62/265,289, filed on Dec. 9, 2015.

(51) Int. Cl. A61B 5/145; C12Q 1/00; C12Q 1/54; G01N 21/65; G01N 33/543; H10F 30/10; H10F 77/40

(52) U.S. Cl. CPC A61B 5/14532; C12Q 1/00; C12Q 1/54; G01N 21/658; G01N 33/54373; H10F 77/496; H10F 30/10

(57) ABSTRACT

Embodiments of the invention are directed to biosensors comprising one or more encapsulated functionalized domains, where the encapsulating matrix acts as the primary interface between the biosensor and the environment. Embodiments of the invention are also directed to the fabrication of the biosensor.

### CROSS-REFERENCES TO RELATED APPLICATIONS

This application is a continuation of U.S. application Ser. No. 16/060,264 filed on Jun. 7, 2018, which is a U.S. national stage application of PCT/US2016/065981 filed on Dec. 9, 2016, which claims priority to and incorporates by reference the entire disclosure of U.S. Provisional Patent Application No. 62/265,289 filed on Dec. 9, 2015.

### STATEMENT REGARDING FEDERALLY SPONSORED RESEARCH OR DEVELOPMENT

This invention was made with government support under CBET-1403002, CBET-0640037 and CMMI-1258696 awarded by the National Science Foundation. The government has certain rights in the invention.

### BACKGROUND OF THE INVENTION

Enzymatic biosensors have been developed for sensing various analytes, including cholesterol, lactate, urea, ethanol, ascorbic acid, bilirubin, choline, glutamine, uric acid and glucose. Wearable biosensors, such as glucose monitors, have been on the market for several years but face many technical, economic, and social challenges in achieving widespread adoption. Some of these challenges include sensitivity loss, requiring daily re-calibration and replacement with a new sensor every few days, a percutaneous connection resulting in patient discomfort, inducing tissue damage due to micromotion, providing a potential pathway for infection, low-analyte-level inaccuracies, which confound detection of life-threatening hypoglycemic events in the context of glucose biosensors, and wide variability in performance among users and even between sensors used by the same person. Another major limitation of approved devices is that they measure a single analyte using a single method and they do not employ redundancy or multimode analysis for error-checking. Current biosensors may also invoke the foreign body response inducing inflammation, biofouling, fibrosis, receding microvasculature, and a barrage of free radicals and degradative enzymes at the sensor-tissue interface. The foreign body response may directly impact the performance of the biosensor. A more innovative approach is required to provide avenues for improving accuracy as well as detecting errors.

### SUMMARY OF THE INVENTION

The biosensor of the claimed invention comprises one or more functionalized domains and an encapsulating matrix that functions as the primary interface between the biosensor and the environment. Some embodiments of the invention may have a plurality of types of domains, while others may have only one. Some embodiments employ redundant and/or inversely related sensing capabilities. The encapsulating matrix is typically comprised of a hydrogel that is crosslinked or otherwise connected to form a continuous structure that disperses and immobilizes the functional domains trapped inside. Some embodiments of the invention make use of surface-enhanced Raman scattering (SERS) and/or luminescent enzymatic sensors; in principle, any optical biosensor approach could be incorporated into the hydrogel-encapsulated domain platform.

The biosensor is formed in a two-step process, where a population of one or more functional domains is fabricated in the presence of the desired functional material. Typically, a thin multilayer film coating is applied to the domains. In the second step, the functional domains are encapsulated in the matrix by mixing them to form a uniform suspension, combining the suspension with the matrix precursor, and trapping the functional domains in the matrix by cross-linking, curing, or freezing.

### BRIEF DESCRIPTION OF THE DRAWINGS

FIG. 1 shows a diagrammatic representation of a method of forming microporous hydrogels in accordance with an embodiment of the claimed invention;

FIG. 2 shows the formation of gold nanoparticles with a surface-immobilized pH-responsive dye in accordance with an embodiment of the claimed invention;

FIG. 3A shows the Raman spectra of sensor materials in accordance with an embodiment of the claimed invention;

FIG. 3B shows the Raman spectra of alginate, PDADMAC, PSS, MES buffer and GDL in accordance with an embodiment of the claimed invention;

FIG. 3C shows the normalized Raman spectra of 8× gold-4-ATP loaded MPA hydrogels at pH 4.0, 5.7 and 7.0 in accordance with an embodiment of the claimed invention;

FIG. 4 portrays the glucose permeation rate (dC/dt) through PAH/PSS bilayers composed of cross-linked PSS-[PDADMAC/PSS]₅-[PAH/PSS]ₙ (⋄), cross-linked PSS-[PDADMAC/PSS]₅-[PSS/PAH/PSS/PDADMAC]ₙ (□), non-cross-linked PSS-[PDADMAC/PSS]₅-[PAH/PSS]ₙ (∘), and the primer coating PSS-[PDADMAC/PSS]₅ alone where n=0 (Δ) shows the change in glucose concentration for cross-linked and non-cross-linked PAH/PSS bilayers in accordance with an embodiment of the claimed invention;

FIG. 5 shows the response of MPAC hydrogels to changing oxygen concentrations in accordance with an embodiment of the claimed invention;

FIG. 6 shows the response of uncrosslinked MPAC hydrogels to changing glucose concentrations in accordance with an embodiment of the claimed invention; and

FIG. 7 shows the response of sensor formulations containing glutaraldehyde cross-linked microdomains in accordance with an embodiment of the claimed invention.

### DESCRIPTION OF EXEMPLARY EMBODIMENTS

Embodiments of the invention are directed to a biosensor within which one or more of smaller regions are encapsulated in a manner that keeps them separated from one another. These regions, or domains, are designed to serve specific functions such as provide color or other optical property, catalyze a chemical reaction, or release a drug. In certain embodiments, the biosensor may contain several of a single type of domain. In other embodiments, the biosensor may contain more than one type of domain, providing several functions that may work independently or in combination. For example, the biosensor may be designed as an implant that has functional domains to drive an oxidation reaction, report oxygen level optically, and release a drug at a controlled rate. The encapsulating matrix acts as the primary interface to the environment, such that compatibility is determined by this material, and it physically maintains the smaller domains in a fixed location, not allowing the domains to escape the confines of the matrix as well as maintain a fixed relative distance between the domains embedded throughout the matrix. In certain embodiments, this may allow for the use of otherwise unusable physical or chemical features due to the smaller scale of their application. The encapsulating matrix then provides a uniform, biocompatible interface and retains the small domains in a constant location.

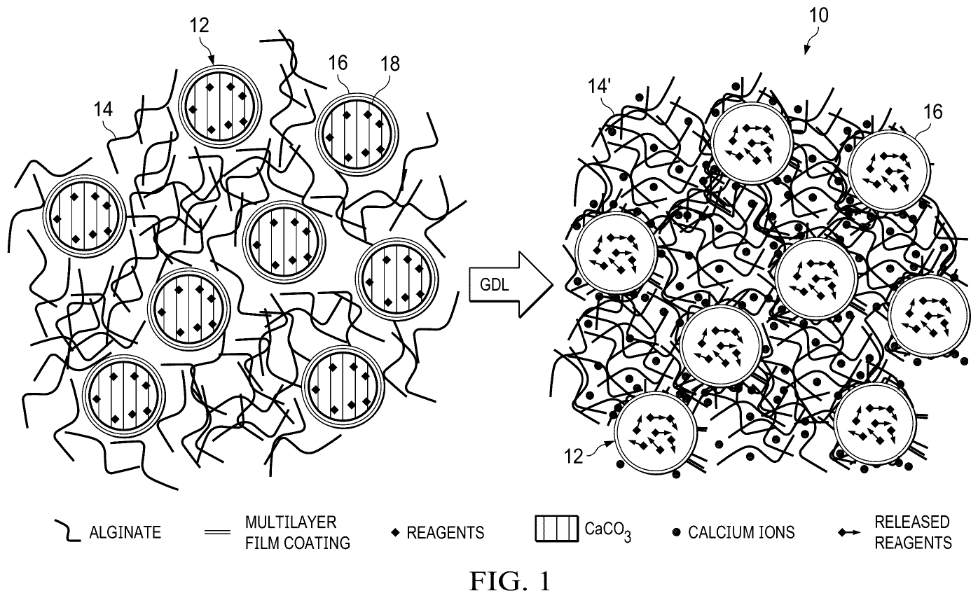

Another embodiment of the invention is directed toward a method of creating and dispersing functional domains in a matrix with desired characteristics. Characteristics of the functional domains and matrix may be altered to match application requirements independently. In certain embodiments, the reactor microdomains may contain large molecules and may require small molecules to diffuse inside quickly, while the drug depot microdomains may contain small molecules that need to be released very slowly. By building these types of microdomains independently, and then incorporating them together into a single encapsulating matrix, a complex multifunctional system may be achieved. FIG. 1 illustrates an example of a biosensor 10 having functional domains 12 dispersed within an encapsulating matrix 14′. First, a population of one or more functional domains 12 is fabricated by forming microspheres or nanoparticles 18 in emulsion or by precipitation from an aqueous solution in the presence of the functional material (reagents). A thin multilayer film coating 16 is then applied to provide the required transport control (pore/mesh size and thickness) for the given encapsulates and functional requirements. These steps may be repeated to produce as many different types of domains 12 as desired. After the number of desired domains 12 is achieved, the functional domains 12 are encapsulated within the encapsulating matrix precursor 14. The functional domains 12 are mixed together to form a uniform suspension. The suspension is then combined with the matrix precursor. Lastly, the encapsulating matrix precursor 14 is crosslinked or otherwise frozen or cured to trap the functional domains 12 in a matrix 14′. In some embodiments, this step may be performed in a mold so that the final product possesses a shape desired for the final application. In the embodiment shown in FIG. 1, the functional domain 12 contains calcium carbonate (CaCO₃) and reagents that are physically trapped in the CaCO₃ when the microparticle is formed. GDL initiates dissolution of CaCO₃ particles, releasing the reagents and Ca²⁺ ions. The released reagents are then mobile and may move freely within the confines of the hollow microsphere having the multilayer films as the boundaries. The latter calcium ions diffuse through the multilayer film coating 16 to interact with the surrounding alginate matrix precursor 14 and induce gelation into a hydrogel comprising ionically-crosslinked alginate matrix 14′. This results in the formation of biosensor 10 comprising an alginate matrix 14′ around functional domains 12 that are hollow microspheres which contain reagents that are free to move within the confines of the microsphere walls.

In an embodiment of the invention, the matrix is produced by reacting a monomer, an initiator, and a crosslinker. In some embodiments, the encapsulated species of the functional domains may be dyes, nucleic acids, proteins, peptides, organic (polymer based) nanoparticles, inorganic (such as gold, silver, or silica) nanoparticles, or small drug molecules.

An embodiment of the invention is directed to a SERS-based pH sensor. A typical SERS-based glucose sensor comprises at least one pH-sensitive acid molecule that is adsorbed on to a gold nanoparticle surface. In certain embodiments, the SERS pH sensor is used in conjunction with an enzyme that drives the pH change to provide a sensor for the enzymatic substrate (e.g. glucose).

A further embodiment of the invention is directed to a phosphorescence-based O₂ sensor. A typical phosphorescence-based O₂ sensor comprises at least one phosphorescent dye fabricated on a suitable template. In certain embodiments, the template used is CaCO₃. The dyes that may be used in these sensors can be any phosphorescent dye. Examples of suitable phosphorescent dyes include Pd-meso-tetra(4-carboxyphenyl) porphine (PdTCPP) and Pd(II) meso-tetra (sulfophenyl) tetrabenzoporphyrin (PdTSTP). While the former is more sensitive to oxygen and may be preferred for high-performance applications when signal levels are not as critical; the latter has a longer (red) excitation and near-infrared emission wavelength and therefore is typically preferred for use in applications wherein light directed to and from the sensor must traverse a highly scattering and/or absorbing medium (e.g. biological tissue). In certain embodiments, the phosphorescence-based O₂ sensor is used in conjunction with an enzyme that drives the O₂ change to provide a sensor for the enzymatic substrate (e.g. glucose).

Another embodiment of the invention is directed to multianalyte and multimodal sensors. In these sensors, multiple pH, oxygen, and/or enzyme substrate (glucose, lactate, etc) sensors are combined into a single device thus allowing for the integration of sensing assays. In certain embodiments, individual sensors that are capable of either pH or O₂ sensing are embedded within a suitable matrix. In other embodiments, sensors that are each capable of sensing multiple analytes or modalities are embedded within a suitable matrix.

In an embodiment of the invention directed to an enzymatic glucose sensor, glucose oxidase (GOx) catalyzes the oxidation of glucose in the presence of molecular oxygen, producing gluconic acid (Glucose+O₂+glucose oxidase+H₂O→gluconic acid+H₂O₂). The decrease in molecular oxygen is proportional to the amount of glucose oxidized. Thus measuring the decrease in molecular oxygen enables the indirect measurement of glucose concentrations. In certain embodiments, an engineered coating is required to drastically reduce glucose diffusion while still allowing molecular oxygen to traverse freely due to lower oxygen concentrations in tissue compared to glucose concentrations, and the implant itself residing in tissue with a significantly decreased oxygen supply. In certain embodiments, a cross-linked polyelectrolyte multilayer (PEM) may be used as an effective diffusion barrier.

In an embodiment of the invention directed to a glucose sensor, glucose oxidase from Aspergillus niger and oxygen-sensitive phosphor such as palladium benzoporphyrin are entrapped within calcium carbonate microparticles via co-precipitation from salt solutions. The microparticles are then encapsulated in a surrounding shell comprising 15 bilayers of poly(sodium 4-styrenesulfonate) and poly(allylamine hydrochloride) (PSS-PAH), which is then crosslinked to reduce pore size, and hence, glucose diffusion. A matrix of hydrogel (e.g. poly(ethylene glycol) (PEG)) is made by dispersing the pre-made sensing capsules in a precursor solution (e.g. PEG-diarylate), crosslinker (e.g. Ethylene glycol dimethacrylate (EGDMA)), and initiator (e.g. Irgacure).

In a similar embodiment of the invention directed to a glucose sensor, glucose oxidase from Aspergillus niger and oxygen-sensitive phosphor such as palladium benzoporphyrin are entrapped within alginate microparticles via emulsion processing. The microparticles are then encapsulated in a surrounding shell comprising 15 bilayers of poly(sodium 4-styrenesulfonate) and poly(allylamine hydrochloride) (PSS-PAH), which is then crosslinked to reduce pore size, and hence, glucose diffusion. A matrix of hydrogel (e.g. calcium-crosslinked alginate) is made by dispersing the pre-made sensing capsules in a precursor solution (e.g. sodium alginate) and supplying the divalent cation crosslinker (e.g. calcium) via external or internal sources.

An embodiment of the invention is directed to a method of creating luminescent enzymatic sensors. Phosphorescent dyes such as PdTCPP (λₑₘ=530 nm, λₑₘ=680 nm, τ₀-1 msec) or PdTSTP may be used as the oxygen indicator. The dye is entrapped with GOx within alginate microspheres prepared from water-in-oil emulsion.

In an embodiment of the invention, a mixture of 3% alginate (pure, high-viscosity, 281 cps), calcium carbonate nanoparticles (4 mol Ca²⁺: 1 mol COO⁻ in alginate), 100 μM GOx, 1 mM albumin, and 1 mM PdTCPP are mixed and dispersed in iso-octane with 1.5% SPAN 85 and 0.75% TWEEN 85 under rapid stirring. Glucone-δ-lactone (GDL) is added to initiate carbonate dissolution (2:1 mol GDL:mol Ca²⁺), resulting in gelation of the droplets upon calcium release. The ionically-crosslinked particles are then harvested by centrifugation, rinsing, and sieving to collect 50-100 μm particles. Rinsing is then performed with pure water with 50 mM CaCl₂. Enzyme and dye are covalently linked to the alginate and one another via N-(3 dimethylaminopropyl)-N′-ethylcarbodiimide hydrochloride (EDC). Polymer coatings are then applied to the particles using layer-by-layer nanoassembly techniques to stabilize the alginate particles and adjust transport. Particles are coated with poly(sodium 4-styrenesulfonate) (PSS, M_{w}=70 kDa) and poly(allylamine hydrochloride) (PAH, M_{w}=50 kDa) through a sequential adsorption process. Particles are briefly suspended in 15 mL of water with 50 mM CaCl₂ and dropped into stirring polyelectrolyte solution (20 mg/mL PAH or PSS in 50 mM CaCl₂) and stirred for 10 minutes. Particles are centrifuged at 500 g for 5 minutes and washed in 30 mL 50 mM CaCl₂), then resuspended in 15 mL buffer. The process is repeated using oppositely-charged polyelectrolyte until 21 bilayers of material have been deposited, resulting in a polymer shell of ˜50 nm with a negative surface charge.

In an embodiment of the invention directed to a method of creating SERS enzymatic sensors, monodisperse 60 nm gold nanoparticles are synthesized using established methods, resulting in CTAB surfactant-stabilized colloid (10% solids). In certain embodiments of the invention, citrate can be used as an alternate for CTAB. These are then treated with HCl by adding 10 M solution until the pH reaches 1.4, resulting in partial removal of the surfactant layer. After exposure for 3 hours at 60° C., the particles are purified by centrifugation at 10000 rpm for 30 minutes and redispersed in deionized water. The particles are then functionalized with the pH responsive pATP or 4-aminothiophenol (4-ATP) by adding 5 μL of 10 mM pATP or 4-ATP in ethanol to 10 mL of aqueous suspension of gold and aging for several hours before use. These nanoprobes are then embedded within alginate microspheres. In certain embodiments of the invention (as discussed below), 4-Mercaptobenzoic acid (MBA), is used as the preferred pH-sensitive molecule as an alternative to 4-ATP.

In an embodiment of the invention directed to a method of synthesizing competitive binding sensors for glucose sensing, gold nanoparticles (60 nm, 10% solids, no surfactant) are suspended in an aqueous solution containing thiolated boronic acid for 72 hours, followed by washing with methanol and finally dispersed in 0.05 M PBS buffer at 10% solids. β-cyclodextrin (βCD) is labelled with Rhodamine 6G (R6G) and titrated into the BA nanoparticle solution until Rhodamine fluorescence rises linearly with each addition, indicating the BA receptors are saturated, yielding data for a baseline assay. The solution is then tested by direct titration of glucose (0, 40, 90, 120, 160, 200, 400 mg/dL) and has both fluorescence and Raman signals measured at each step. Nanoparticle size, boronic acid density, competing ligand type, and the relative concentrations are then adjusted from the baseline as needed to achieve a response over the desired glucose range.

Another embodiment of the invention is directed to multimodal, redundant optical sensing with intrinsic error-checking; multimodal meaning dual detection modes as well as dual transduction modes. Such sensors may simultaneously produce both fluorescence and SERS signals. Luminescence and SERS signals that vary directly and inversely with analyte concentration, respectively, provide complementary design capable of checking within each transduction mode. Sensors with regions based on different transduction schemes (affinity vs. enzymatic catalysis) yield different sensitivities and allow for error-checking between transduction modes. Redundant sensors allow signal averaging to improve accuracy and the use of fault detection algorithms to detect the failure of individual array elements.

In certain embodiments of the invention, Concanavalin A (ConA) and deactivated GOx (apo-GOx) are used as affinity receptors. These sensors have the advantages that they do not consume glucose (or oxygen) or produce any byproducts and they are purely sensitive to concentration (not analyte flux). This makes them highly complementary to and presumably more reliable than the enzymatic sensors of the other embodiments. However, because the sensitivity is relatively low and the binding is not highly selective, they are still susceptible to other types of nonspecific signal changes—but, they are influenced differently than the enzymatic types. For example, effects of photobleaching are particularly problematic in these systems. To address this, an attractive enhancement is to use a separate optical modality (e.g. SERS) with opposite sensitivity for error checking. In certain embodiments, sensors may be based on ConA or boronic acid (BA) receptors attached to gold nanoparticles. The competing ligand may be β-cyclodextrin (βCD), which has been reported to compete with glucose for binding ConA and boronic acid. βCD can be labelled with Rhodamine 6G (R6G), a strong fluorophore and well-known Raman reporter dye, allowing these reagents to serve as both fluorescent and Raman signal sources, without the need for separate chemistries. In the low-glucose state (βCD bound to BA), the gold may quench fluorescence from R6G while simultaneously amplifying the Raman scattering from the dye. Conversely, as glucose concentration increases, displacement may result in unquenching of fluorescence and a corresponding loss of SERS signal.

In an embodiment of the invention directed to a multimodal optical bread board based system a 530 nm laser source is combined with two small, hand-held spectrometers on a single optical bread board using a trifurcated multifiber system. The fiber probe is constructed with a single center fiber for excitation delivery, and several collection fibers around the outside, half of which are coupled into the Raman system and the other half into the fluorescence system. The light from the sample is collected by the fibers filtered as appropriate, and focused onto the slit of a respective Raman and fluorescence spectrometers, and measured by a CCD array in each case. This allows for real-time collection of the signal simultaneously at all wavelengths within the band of interest. Having multiple wavelengths provides for utilization of post-processing algorithms such as partial least squares, as needed, to correlate the intensity change in an analyte (such as glucose) concentration. Other embodiments of this apparatus may be significantly smaller. Performance specifications are determined by using standard luminophores and SERS reporters (R6G, R6G-gold, pATP-gold). The signals from these materials are measured in aqueous buffer, then through silicone skin phantoms with different thickness to quantify signal-to-noise relationships with depth. Finally measurements of sensors inserted at varying depths. (0.5-4 mm) in live animals (rats and pigs) may be used to gauge the effects of tissue and path length variations on signal strength and spectrum. In certain embodiments, where signal processing such as spectral derivatives, de-trending, Standard Normal Variate (SNV), and Multiplicative Scatter Correction (MSC) fails to elucidate the Raman spectra, a second, long wavelength laser (785 nm) may be used to reduce the fluorescence contribution to the Raman spectra. By adding a second center fiber, the two signals may still be detected simultaneously. In other embodiments, the lasers can be time multiplexed to avoid optical bleed-through from one wavelength system to the other.

In an embodiment of the invention directed to a method of encapsulating reagents in polymer microcapsules, reagents are encapsulated within hollow polyelectrolyte capsules. Fresh baseline assay solutions are prepared in 8 mL of 0.25 M Na₂CO₃. Each of these is then mixed with 8 ml of 0.25 M CaCl₂ solution under rapid stirring. After 30 seconds, stirring is ceased and the particles mature under static conditions for 10 minutes. Following the centrifugation at 500 g for 5 minutes, the particles are then washed with buffer three times. Polymer coatings are then applied to the microparticles with entrapped sensing chemistry using layer-by-later nanoassembly techniques using 50 mM Tris buffer (pH 8.5).

In an embodiment of the invention directed to a method of distributing capsules in molded alginate hydrogels, microporous hydrogels are formed by combining the PEM-coated calcium carbonate microspheres with 2% alginate (ultrapure, high-G, ProNova) and glucone-δ-lactone (CDL). GDL initiates dissolution of CaCO₃ particles, releasing the sensing reagents and Ca²⁺ ions, the latter of which diffuse through the polyelectrolyte films to ionically crosslink the surrounding alginate into a hydrogel. This results in the formation of alginate around hollow pockets of sensing reagents. The molar ratio of CaCO₃ to CDL is fixed at 1:2, while the relative amounts of CaCO₃ and GDL to alginate is varied to observe the resulting effect on hydrogel mechanics and sensor response, particularly sensitivity, range, and response time.

In an embodiment of the invention directed to a method of fabricating sensing gels, disc-type sensors are prepared as 750 um thick alginate slabs by casting the gels in a Teflon mold with a glass lid. Biopsy punches (2.5 mm diameter) are used to remove the samples. In certain embodiments, samples may be immobilized on a glass slide for testing.

An embodiment of the invention is directed to a method of characterizing the response of each sensor type, in which a specialized automated testing apparatus may be utilized to characterize sensor responses. In certain embodiments lacking a dual-mode optical system, a bifurcated optical fiber bundle with input and output arms combined into a single probe end are used to measure either fluorescence or Raman spectra by interfering with the respective spectrometer individually. For fluorescence, excitation light is delivered from a green LED (with 500±5 nm filter) and collected emission light is monitored with an array spectrometer (such as USB 2000, Ocean Optics; range: 500-800 nm). For Raman spectroscopy, a fiber-coupled Raman system may be used (such as an Ocean Optics R-300). Characterizations are determined by step response tests and static stability tests. Response stability over time is determined by static stability tests.

A further embodiment of the invention is directed to a method of fabricating a multizone implant, in which a slow-gelling formulation is developed to enable sequential deposition of viscous precursors into a mold, followed by homogenous crosslinking to achieve discrete regions with different chemistry. Precursors of both assay types are prepared by first producing the respective PEM-coated calcium carbonate microspheres with encapsulated microspheres/sensor chemistry. These are then combined with 1-3% alginate to yield two different hydrogel precursors. The precursors are then dispensed into a 2.5 mm-i.d. Teflon mold by 20 uL increments, such that four layers are present. Each 20 uL adds ˜1 mm of height; sensor samples are prepared with region thickness varying from 2.5 to 5 mm. In certain embodiments, spacer layers without sensing chemistry may be inserted in some samples to evaluate independent addressability. Alginate concentration is chosen to avoid mixing prior to gelation. GDL is introduced immediately before or after dispensing, depending upon the observed. CaCO₃ dissolution rate. In certain embodiments, steps may be modified to achieve contiguous stable hydrogels with discrete sensing regions.

An embodiment of the claimed invention is directed to an optical sensing platform that is designed to facilitate noninvasive measurements of metabolic data in various culture and animal models with dramatically improved temporal resolution. Integrating the sensor materials of the claimed invention into the culture or tissue and using noninvasive optical interrogation overcomes the limitations of prior art approaches.

Applications of the sensors of the claimed invention include broad value to biosensing technology in three areas: (1) improving the sensitivity and operation in the presence of larger molecules (e.g. proteins); (2) expanding the application of SERS assays from single-use to reversible monitoring systems and (3) development of portable, low-cost, miniature Raman sensing systems.

Embodiments of the invention are directed to the bimodal sensing of pH and oxygen in a nano-composite hydrogel based sensor. Characteristic Raman scattering peaks attributed by carboxyl and carbonyl groups present on the Raman sensitive molecule 4-MBA are sensitive to pH changes making them candidates for use in pH sensing. Whereas, metallo porphyrin phosphorescent dyes, which are easily quenched by the presence of molecular oxygen, has been predominantly used in optical oxygen sensors. Utilizing a pH sensitive Raman molecule and an oxygen sensitive phosphorescent dye, a microporated alginate hydrogel, containing discrete pH sensing and oxygen sensing micro-domains can be constructed. The pH sensing micro-domains contain surface enhanced Raman scattering (SERS) active MBA capped gold nanoparticles (AuNPs) and the oxygen sensing micro-domains contain a phosphorescent dye Pd (II) meso-Tetra (sulfophenyl) Tetrabenzoporphyrin (PDTSTPPdTSTP). Polymeric microcapsules capable of sensing either pH or oxygen concentrations are made by taking advantage of the well-established layer-by-layer (LbL) protocol. Encapsulating the pH sensing and oxygen sensing components into segregated micro-domains distributed in an alginate hydrogel both pH and oxygen to be sensed and measured using dual sensing modalities.

### WORKING EXAMPLES

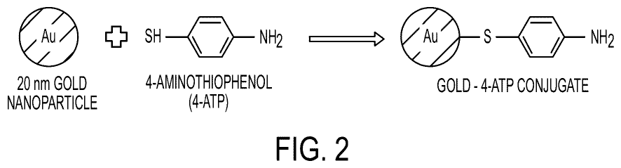

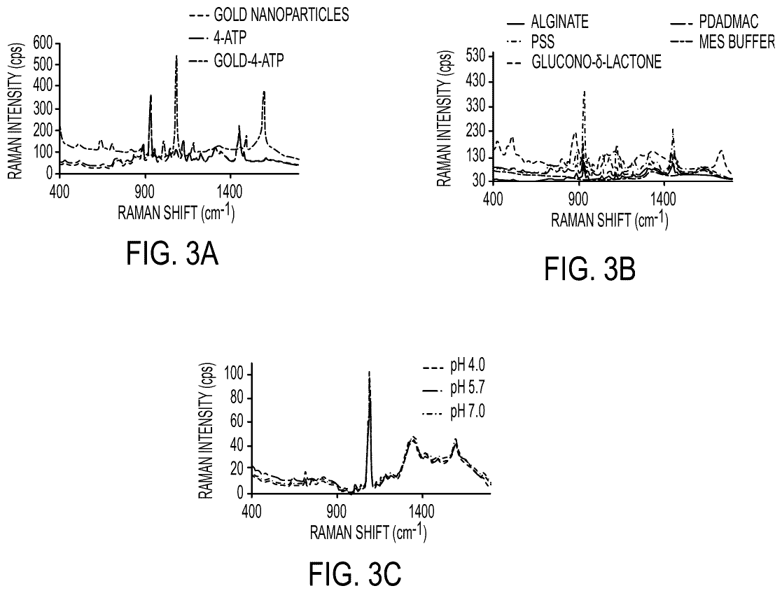

CaCO₃ particles were co-precipitated with GOx and PdTCPP in 19.5-bilayer PSS/PAH capsules. Reagents were encapsulated within hollow polyelectrolyte capsules. Fresh baseline assay solutions were prepared in 8 mL of 0.25 M Na₂CO₃. Each of these was then mixed with 8 ml of 0.25 M CaCl₂ solution under rapid stirring. After 30 seconds, stirring was ceased and the particles matured under static conditions for 10 minutes. Following the centrifugation at 500 g for 5 minutes, the particles were then washed with buffer three times. Polymer coatings were applied to the microparticles with entrapped sensing chemistry using layer-by-layer nanoassembly techniques using 50 mM Tris buffer (pH 8.5). Additional particles co-precipitated with FITC-labeled GOx were prepared for imaging capsule distribution in hydrogels. Polymer coatings were also applied although a fraction of particles w % ere left uncoated to prepare control alginate hydrogels. Three hydrogels were then formed by combining the calcium carbonate microspheres with 2% alginate and GDL, casting each mixture into a 0.75″×1.5″×750 um Teflon mold with glass lid. A 2.5 mm biopsy punch w % as used to extract a sample, which was then subjected to response testing by measuring either fluorescence or Raman spectra. For fluorescence, excitation light was delivered from a green LED (with 500±5 nm filter) and collected emission light is monitored with an array spectrometer (USB 2000, Ocean Optics; range: 500-800 nm). For Raman spectroscopy, a fiber-coupled Raman system was used (Ocean Optics R-300). As can be seen from the SEM images, the coated particles result in the desired hollow pocket architecture; the control gel displays a smooth surface with folds resulting from drying for imaging. Confocal analysis of a gel with labeled enzyme revealed discrete domains (“pockets”) distributed throughout the gel and compartmentalizing the sensing chemistry. In response to step increases in glucose concentration, phosphorescence measurements of two different samples made with the same formulation exhibited a sensitive, repeatable, and consistent response. These data demonstrate the feasibility of these approaches to encapsulation and hydrogel integration and demonstrate that these approaches are sufficiently gentle to preserve function of the sensing chemistry. A stratified alginate hydrogel was fabricated by a slow-gelling formulation developed to enable sequential deposition of viscous precursors into a plastic cuvette mold, followed by homogenous crosslinking to achieve discrete regions with different chemistry. Three precursors were prepared using carbonate particles coprecipitated with yellow-green (G) and red (R) polystyrene particles and yellow quantum dots (Y). GDL was added, then alginate was dispensed with approximately 1.3 mL aliquots into a 1 cm plastic cuvette (Order: G/Y/R/Y/G/Y/R). The cuvette was placed on a UV transilluminator and a photograph was taken to illustrate the discrete sensing regions. This demonstrates the feasibility of the proposed redundant sensor fabrication process. In addition, alginate hydrogels were prepared with microencapsulated gold nanoparticles with a surface-immobilized pH-responsive dye. The pH-sensitive molecule 4-ATP (97%, Sigma-Aldrich) was dissolved in pure ethanol at a concentration of 0.5 mg/mL, and incubated with 20 nm gold nanoparticles (citrate capped, aqueous, obtained from Nanopartz, Inc.) overnight at a 1:1 ratio by volume (FIG. 2). After sonication for one hour, this solution of gold-4-ATP was purified with a 30 kD Nanosep filter by centrifugation at 5,000 g, washing with both pure ethanol and 18.2 MΩ-cm deionized water (Pall Cascada LS). Loading of CaCO₃ microspheres with gold-4-ATP followed the adapted CaCO₃ microspheres synthesis protocol. Briefly, 400 uL of gold-4-ATP was added to 6 ml of 0.20 M Na₂CO₃ at room temperature. 6 mL of 0.20 M CaCl₂ was added to this solution under vigorous stirring for 30 seconds, and then allowed to sit for 10 minutes. After centrifuging the solution and removing the supernatant, the packed particles were resuspended in 750 ul of a 5 mM NaHCO₃ buffer at pH 8.0. Immediately after synthesis, the microspheres were coated with a series of oppositely charged polyelectrolytes. Poly(diallyldimethylammonium chloride) (PDADMAC, obtained from Sigma-Aldrich) was first deposited onto the surface of these CaCO₃ microspheres by incubating them in a 20 mg/ml solution of the polyelectrolyte for 30 seconds, with moderate mixing. Poly(sodium 4-styrenesulfonate) (PSS, obtained from Sigma-Aldrich) was then deposited in an identical fashion, and successive layers of PDADMAC and PSS were alternated until a total of 10 layers (5 bilayers) was reached. The microspheres were washed once with a 5 mM NaHCO₃ buffer at pH 8.0 between each layer and at the end of the process. Microporous alginate composite (MPA) hydrogels with three different concentrations of gold-4-ATP loaded CaCO₃ microspheres were fabricated following the steps described above. To synthesize a “3×” concentration MPA hydrogel (with a ratio of 1:0.27:2 of CaCO₃:carboxylic acid (from alginate):glucone-δ-lactone), 2.55 mg of gold-4-ATP loaded CaCO₃ microcapsules was washed and resuspended in 25 uL of deionized water. This was added to 50 uL of 3% w/v sodium alginate solution (alginic acid sodium salt from brown algae, obtained from Sigma-Aldrich). 25 uL of 200 mg/ml glucone-δ-lactone was then added, and the solution allowed to fully gel for one hour. Similarly, a “5×” concentration MPA hydrogel was synthesized using the same procedure above, but instead using 4.26 mg of gold-4-ATP loaded CaCO₃ microspheres and 25 uL of glucone-δ-lactone at 333 mg/mL. An 8× concentration MPA hydrogel was likewise synthesized using 6.80 mg of gold-4-ATP loaded CaCO₃ microspheres and 25 uL of glucone-δ-lactone at 533 mg/mL. After gelation, the hydrogels were washed three times in a 10 mM MES buffer with 10 mM CaCl₂) at pH 5.7. Thermo Scientific DXR Raman microscope was used to perform Raman spectroscopy on the prepared MPA hydrogels loaded with gold-4˜ATP and the various controls, at room temperature. A 780 nm laser was used to irradiate the samples at a power intensity of 20 mW through a 50 um aperture, with a grating of 830 lines/mm; 120 exposures were taken per sample, at an exposure time of 3 seconds. FIG. 3A portrays the Raman spectra of sensor materials in the top left, FIG. 3B portrays the Raman spectra of alginate, PDADMAC, PSS, MES buffer, and GDL. FIG. 3C portrays normalized Raman spectra of 8× gold-4-ATP loaded MPA hydrogels at pH 4.0, 5.7, and 7.0. The spectra (FIG. 3A) from the sensor materials reveal distinctive peaks for the 4˜ATP conjugated nanoparticles. These do not overlap strongly with the SERS bands for any of the other components of the proposed sensor devices, and clearly appear in the spectra from the full hydrogel system. These data support the concept of using microencapsulated SERS probes embedded in alginate matrix.

### Experimental Section

### Chemicals

Sodium carbonate (Na₂CO₃), calcium chloride (CaCl₂)), poly (sodium 4-styrenesulfonate) (PSS, average Mw 70000 Da), poly (diallyldimethylammonium chloride) (PDADMAC, average Mw 100000-200000 Da), poly(allylamine hydrochloride) (PAH, average Mw 15000 Da), glutaraldehyde solution (grade II, 25% in H₂O), alginic acid sodium salt from brown algae (100-300 cP, 2% at 25° C.), and buffer salts (NaHCO₃, MES and TRIS) were obtained from Sigma and were used as received without further purification. Glucose oxidase (GOx) from Aspergillus niger (257 U/mg, BBI enzymes) and Pd-meso-tetra (4-carboxyphenyl) porphyrine (PdTCPP, Frontier Scientific) suspended in DMSO (10 mM) solution were used in all experiments. Glucose used for all sensor response studies was obtained from Macron Fine Chemicals™

### Layer-by-Layer Assembly on Planar Substrate

Nanofilms were deposited on Anopore™ inorganic aluminum oxide membrane filters (dia. 25 mm, pore size 0.02 μm, Sigma) placed in an open-face filter holder (Pall Co.). The open face of the filter membrane was exposed to oppositely charged polyelectrolyte solutions (20 mg/ml PDADMAC (pH 8), 20 mg/ml PAH (pH 8), 20 mg/ml PSS (pH 7.2) alternately with wash steps (5 mM NaHCO₃) between each polyelectrolyte exposure step. A primer coating consisting of [PSS]-[PDADMAC/PSS]₅ was deposited to achieve complete surface coverage before depositing the desired number of PAH/PSS bilayers. After depositing the target number of PAH/PSS bilayers, the nanofilms were exposed to 0.1 M glutaraldehyde solution for 30 minutes to cross-link the amine groups on PAH. Excess glutaraldehyde was removed by washing the nanofilms with 5 mM NaHCO₃ (pH 7.2).

To fabricate interspersed cross-linked PAH layers, a PSS/PDADMAC layer was deposited between successive PAH/PSS bilayers. Cross-linking of the interspersed layers was performed using the same protocol to cross-link non-interspersed PAH/PSS bilayers. When depositing PAH/PSS bilayers wash steps were performed using 5 mM NaHCO₃ (pH 7.2), and while depositing PDADMAC/PSS bilayers 5 mM NaHCO₃ (pH 8) was used for the washing steps, to ensure that the polyelectrolytes were sufficiently ionized while being deposited.

### Diffusion Measurements

Nanofilms fabricated on Anopore™ membrane filters, were placed between the feed and the permeate chambers of a side-by-side diffusion cell (Permegear Inc.). The feed chamber was filled with 7 ml of 5 mM NaHCO₃ (pH 7.2) containing 1 g/l glucose and the permeate chamber was filled with 7 ml of 5 mM NaHCO₃ (pH 7.2). Samples were collected from both the feed and the permeate sides at regular time intervals, and the glucose concentration of the samples were measured using a YSI biochemistry analyzer (2700 Select). The slope of the concentration increase over time in the permeate chamber (dC/dt) was calculated by linear regression for the different nanofilm formulations.

Nanofilm-Coated Microparticles with Encapsulated Sensing Chemistry

PdTCPP and GOx containing calcium carbonate (CaCO₃) microparticles were synthesized using the co-precipitation method, with minor modifications. Briefly, 200 μl of 10 mM PdTCPP solution was added to 8 ml of 0.2 M Na₂CO₃ containing 64 mg of GOx under continuous stirring (800 RPM). After 5 mins, 8 ml of 0.2 M CaCl₂) was added rapidly and the reaction was allowed to continue for 10 mins. Nanofilms were deposited on the PdTCPP and GOx containing microparticles, by alternately exposing the particles to polyelectrolyte solutions (20 mg/ml PDADMAC (pH 8), 20 mg/ml PAH (pH 8), 20 mg/ml PSS (pH 7.2)) with intermediate wash steps. The wash solutions used were the same as described above for making nanofilms on planar substrates. After depositing the desired number of nanofilms, 3.3 mg of nanofilm-coated microparticles was suspended in 10 ml, 0.3 M glutaraldehyde solution for 30 min. Excess glutaraldehyde was removed by washing the microparticles with 5 mM NaHCO₃ (pH 7.2). The amount of glutaraldehyde used for the microcapsules was based on the ratio of [nanofilm surface area]:[mass of glutaraldehyde].

### Microporous Alginate Composite (MPAC) Hydrogels

MPAC hydrogels were made using the protocol described herein. Briefly, PEM coated CaCO₃ microparticles (3.3 mg suspended in 100 μl of deionized water), 3% alginate solution (200 μl) and GDL (100 μl of 133 mg/ml) were mixed to make a slow-gelling hydrogel precursor. The precursor was then poured between two glass slides separated by a 0.06″ Teflon spacer, and allowed to gel for 24 hours.

### Characterization

Confocal fluorescence and differential interference contrast (DIC) microscopy images were captured using an inverted laser spinning-disk confocal microscope (Olympus IX81). Hydrogel samples excited at 488 nm were viewed through 40× and 100× oil immersion objectives. Images were analyzed using Image J software.

SEM images of nanofilm coated microparticles, microcapsules and MPAC hydrogels were captured using a JEOL 7500 scanning electron microscope. A diluted sample of either nanofilm coated microparticles or microcapsules was placed on a silica wafer and was allowed to dry overnight. For imaging, microcapsules were made by exposing the microparticles to 10 ml of 0.2 M MES buffer (pH 5.8) for 30 minutes. To prepare a hydrogel sample for SEM imaging, a 5 mm×5 mm hydrogel was placed on a silica wafer and dried overnight. All samples were sputter-coated with 2.5 nm of palladium/platinum before imaging.

### Sensor Fabrication and Testing

Hydrogel discs having a diameter of 3 mm were excised from the hydrogel slab using a biopsy punch. Each sample was placed in a liquid flow cell, and changes in lifetime with varied glucose and oxygen concentrations were recorded using a custom time-domain lifetime measurement system.

The response to oxygen was evaluated by flowing buffer having varied dissolved oxygen concentrations (0-206.8 μM). The dissolved oxygen concentration of 10 mM TRIS (pH 7.2) containing 10 mM CaCl₂) was varied by purging air and nitrogen with mass flow controllers (type 1179A, MKS).

To determine the response to glucose, solutions containing different concentrations of glucose (0-400 mg/dl) dissolved in 10 mM TRIS (pH 7.2) with 10 mM CaCl₂) were flowed over the hydrogel samples. The response parameters were calculated from each of the obtained response curves. The limit of detection (LOD) was estimated by calculating the glucose concentration at which the response was 10% higher than the response at 0 mg/dl glucose concentration. Similarly, the maximum differentiable glucose concentration (MDGC) was estimated by calculating the glucose concentration at which the response was 10% lower than response at 400 mg/dl glucose concentration. The range of the sensor was defined as R=MDGC-LOD, while the sensitivity was defined as the percent change in the maximum and minimum response observed per unit range of the sensor.

An embodiment of the invention is directed to constructing a pH sensor, wherein the sensor comprises a pH sensitive Raman molecule. The testing of pH sensors was carried out by analyzing SERS signals from gold nanoparticles coated with mercaptobenzoic acid (MBA-AuNPs). SERS signals were obtained of MBA-AuNPs as a function of pH (4, 5.5, 6, 6.5, 7 and 8.5) via a DXR Raman confocal microscope (Thermo Scientific, Waltham, MA, USA). 50 μL aliquot of MBA-AuNPs stock was mixed with 1.5 mL of 10 mM MES buffer at desired pH. The mixed solution was centrifuged at 2000 g for 15 min. After supernatant removal precipitates on the bottom of centrifuge tube were redispersed in 30 μL of the same buffer solution and then injected into a well of a 384 well plate. The MBA-AuNPs solution was excited by 780 nm laser with the power of 20 mW. Raman scattered light was collected using a 10× objective lens (M Plan, Olympus cooperation, Tokyo, Japan). During the SERS measurement the laser was focused on the solution surface. Five SERS spectra were recorded and averaged from one sample with a collection time of 3 sec. For the resulting spectra background subtraction was performed by a software, CrystalSleuth. The AuNPs described above were incorporated into CaCO₃ microparticles to create AuNP-containing capsules. These microparticles can be incorporated into hydrogels by mixing the particles with alginate and GDL.

An embodiment of the invention is directed to constructing a O₂ sensor, wherein the sensor comprises a phosphorescent dye. In order to measure O₂, micro-capsules containing a phosphorescent dye was fabricated using CaCO₃ as the template. Oxygen quenchable phosphorescent dyes such as PdTCPP and PdTSTP are used in these microcapsules. CaCO₃ micro-particles were co-precipitated with either PdTCPP or PdTSTP, followed by polyelectrolyte multi-layer coating. Particles were coated with [PDADMAC/PSS]₅-[PAH-PSS]₅. After coating the particles with nano-films, the nano-film coated particles were exposed to 0.2 M MES buffer (pH 5.8) to obtain micro-capsules. Similar to the pH sensors, PdTCPP/PdTSTP containing micro-capsules were immobilized in 1.5% alginate hydrogels, and exposed to varying concentrations of oxygen using mass flow controllers.

A further embodiment of the invention is directed to a device comprising integrated sensors that are capable of measuring pH and O₂. In order to test the capabilities of integrated sensors, alginate hydrogels containing pH-sensing micro-capsules and oxygen-sensing micro-capsules were fabricated. Evaluating the behavior of hydrogels containing capsules containing phosphorescent dye and MBA-AuNPs was necessary in order to determine that there was no unwanted interference in either of the sensing modes. Measurements were performed on samples taken from a single slab of the hydrogels, using the same procedures described in the above sections. The phosphorescence behavior was found to be consistent that observed for with otherwise identical hydrogels without the AuNPs, i.e., pH sensors. For the SERS measurements, a relatively high background due to phosphorescent dyes was seen; however, pH calibration curves presented a similar trend with MPAC hydrogel. Thus, these initial data support the feasibility of the planned future studies with combined multimodal assays.

### Effects of Crosslinking as a Diffusion-Limiter

The effect of glutaraldehyde cross-linking of PSS/PAH bilayers on the diffusion of glucose was evaluated by measuring the rate of glucose diffusion across PSS/PAH nanofilm constructs deposited on Anapore™ membranes. An initial primer coating of PSS-[PDADMAC/PSS]s was first deposited to ensure complete surface coverage of the Anapore™ substrate.

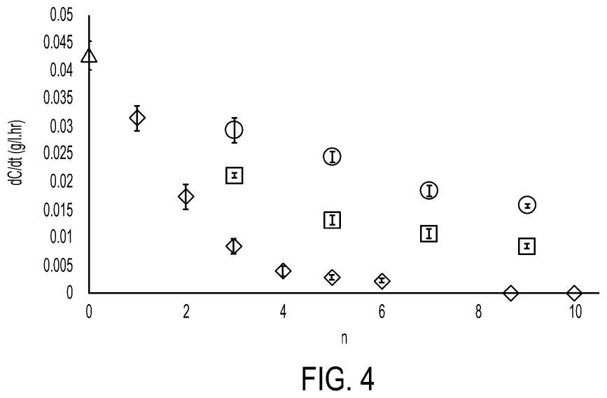

PAH/PSS bilayers were deposited on the primer coating (PSS-[PDADMAC/PSS]₅) to fabricate PSS-[PDADMAC/PSS]₅-[PAH/PSS]ₙ multilayers, where n was varied from 1 to 10. The glucose diffusion across different nanofilm formulations was evaluated by calculating the linear slope of the glucose concentration change dC/dt (where C is the concentration of glucose (g/l) and t is time (hours) on the permeate side of the diffusion cell. The data presented in FIG. 4 shows the decrease in dC/dt for both the cross-linked and non-cross-linked PAH/PSS bilayers as the number of layers is increased. FIG. 4 portrays the glucose permeation rate (dC/dt) through PAH/PSS bilayers composed of cross-linked PSS-[PDADMAC/PSS]₅-[PAH/PSS]ₙ (⋄), cross-linked PSS-[PDADMAC/PSS]₅-[PSS/PAH/PSS/PDADMAC]ₙ (□), non-cross-linked PSS-[PDADMAC/PSS]₅-[PAH/PSS]ₙ (∘), and the primer coating PSS-[PDADMAC/PSS]s alone where n=0 (Δ). Error bars represent 95% confidence intervals for three separate nanofilm constructs. It is quite clear that the decrease in dC/dt is much more pronounced in the case of the cross-linked PAH/PSS bilayers. Specifically, the glucose permeation rate through non-cross-linked PAH/PSS bilayers decreases by ≈46% when n is increased from 3 to 9, whereas the dC/dt of cross-linked PAH/PSS bilayers decreases by ≈98% for the same number of bilayers. It is evident that the cross-linked films more effectively prohibit the free diffusion of glucose compared to the native nanofilm constructs. For the same number of bilayers, cross-linking significantly decreases the dC/dt across the multilayer constructs. Comparing the glucose permeation rate through cross-linked and non-cross-linked PEMs when n=3, 5 and 9, the dC/dt of glucose through the cross-linked PEMs was found to be less than the corresponding non-cross-linked PEMs by ≈71%, ≈88% and ≈99%, respectively. It is interesting to observe that the first five cross-linked bilayers decrease the glucose permeation rate drastically; however, further increase in the number of cross-linked bilayers has much less change. The dC/dt values for glucose through the cross-linked PEMs change by ≈39% when comparing diffusion rates between n=1 and n=2, whereas the decrease was only ≈15% when comparing n=5 and n=6.

The drastic decrease in permeability to glucose when the —NH₂ groups of the PAH layer are cross-linked in the presence of glutaraldehyde may be attributed to the decrease in free volume present in the PEMs. Apart from crosslinking the —NH₂ groups of PAH in an individual PAH layer, the possibility also exists to have cross-linked —NH₂ groups present in successive PAH layers due to the interpenetrating nature of layer-by-layer assembled PEMs.

To investigate the effect of interlayer and intralayer cross-linking on glucose diffusion, nanofilms were designed with a spacer bilayer [PSS/PDADMAC] introduced between successive [PSS/PAH] bilayers. The spacer containing PEMs fabricated are represented by PSS-[PDADMAC/PSS]₅-[PSS/PAH/PSS/PDADMAC]ₙ. The spacer containing cross-linked PSS/PAH bilayers were found to limit glucose diffusion to a greater extent than non-cross-linked nanofilms; however, they were also found to be less effective in restricting the diffusion of glucose than the cross-linked PSS/PAH nanofilms which do not contain spacer bilayers (FIG. 4). Introduction of the PSS/PDADMAC spacer bilayer allowed glucose to diffuse through the nanofilm coatings more freely as compared to glucose diffusion across non-spacer containing successively cross-linked films with the same number of PAH layers. For n=3, 5 and 9 the dC/dt of cross-linked PSS-[PDADMAC/PSS]₅-[PSS/PAH/PSS/PDADMAC]ₙ was 2.5, 4.5 and 81 times greater, respectively, than their cross-linked counterparts without the spacers (PSS-[PDADMAC/PSS]₅-[PAH/PSS]ₙ). It is important to recognize that the metrics used are of glucose permeation rate and are not normalized by film thickness. Thus, even though the cross-linked spacer-containing PEMs contain more layers and are overall thicker, the total glucose diffusion barrier is less than the cross-linked PEMs without the spacer bilayers. This increase in dC/dt is ascribed to the reduced interlayer cross-linking by the introduced spacer bilayer that decreases the interpenetration of neighboring PAH layers.

Once the glucose permeation rate effects were determined, the developed planar multilayer scheme was translated to microparticle templates to fabricate microcapsule glucose sensors. The expectation was that the varying glucose permeation rate of the different nanofilms would result in correspondingly shifted glucose sensor behavior (sensitivity and response range). The microparticles and capsules were first characterized by optical and electron microscopy to confirm the desired products were produced in the fabrication process.

Microporated hydrogels with discrete spherical domains containing GOx and PdTCPP function as glucose sensors. As glucose is flowed over the hydrogels, glucose diffuses easily through the alginate, which has a diffusion coefficient similar to water. As glucose diffuses into the microcapsules, the GOx contained in the hydrogel microdomains oxidizes the glucose, and reduces local oxygen concentration proportional to the glucose permeation rate. Hence, changes in glucose concentrations can be determined by optically monitoring the decrease in oxygen concentration. It is imperative to understand that for the glucose sensor to function effectively, the influx of glucose must be balanced to the reaction kinetics of the enzyme (GOx) as well as the supply of oxygen. By increasing the diffusion barrier to glucose, it is intended to create a system that is truly glucose-diffusion limited. Glucose-limited behavior is only achieved if the influx of oxygen is much higher than or equivalent to the influx of glucose.

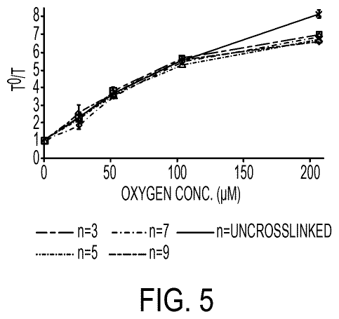

The response of the MPAC hydrogels containing PdTCPP and GOx loaded micro domains to changing oxygen concentrations was evaluated to ascertain whether cross-linking of PAH/PSS bilayers affects oxygen diffusion. As a control, the oxygen sensor response of MPAC hydrogels containing non-cross-linked [PDADMAC/PSS]₅-[PAH/PSS]₉ microcapsules was also determined. FIG. 5 represents the lifetime (normalized to the lifetime at zero oxygen concentration) against varying oxygen concentrations. The cross-linked nanofilm architectures are represented by [PDADMAC/PSS]₅-[PAH/PSS]ₙ where n=3, n=5, n=7, n=9 and uncross-linked nanofilm architecture [PDADMAC/PSS]₅-[PAH/PSS]9. Error bars represent 95% confidence intervals for three separate MPAC hydrogels. The dashed lines are provided only as a guide to the eyes. Using the Stem-Volmer equation τ₀/τ=1+K_{SV} [O₂], the K_{SV} values for the MPAC hydrogels was determined using linear least-squares regression. The K_{SV} values calculated for the different MPAC hydrogels were 0.030±0.002 μM⁻¹, with no significant difference for the samples prepared with different nanofilms (α=0.05). All the hydrogel samples having different nanofilm compositions showed a high sensitivity to oxygen at levels less than 100 μM, and a decreased sensitivity at higher oxygen concentrations, characteristic of oxygen-sensitive palladium porphyrin dyes. The similar oxygen response characteristics show that cross-linking of the nanofilms in the hydrogel does not affect the kinetics of oxygen diffusion.

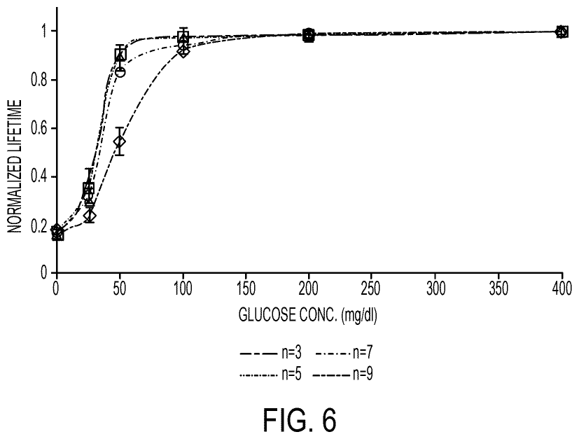

The glucose sensing characteristics of MPACs containing non-cross-linked [PDADMAC/PSS]₅-[PAH/PSS]ₙ nanofilm bounded micro domains were examined to establish that cross-linking of PAH/PSS bilayers was necessary to alter the sensor characteristics significantly. The phosphorescence lifetime of MPAC hydrogels containing PdTCPP and GOx loaded microdomains was recorded as the materials were exposed to buffer solutions containing varied concentrations of glucose (0-400 mg/dl). The sensor response curves for MPACs containing non-cross-linked [PDADMAC/PSS]₅-[PAH/PSS]ₙ nanofilm bounded micro domains are illustrated in FIG. 6, where the change in lifetime is calculated relative to the lifetime obtained at maximum glucose concentration. Error bars represent 95% confidence intervals for three separate MPAC hydrogels. The dashed lines are provided only as a guide to the eyes. A coherent trend was observed in terms of sensitivity and range of the sensors as the number of bilayers was increased. This was anticipated since altering the transport properties of the microcapsule directly influences the sensor characteristics.

With an increase in the number of PSS/PAH bilayers from n=3 to n=9, the analytical range increases by ≈106% while the sensitivity over the same range decreases by ≈59%. This inverse relationship between range and sensitivity is characteristic of flux-based sensors. Table 1 summarizes the sensor parameters for non-cross-linked microcapsule-containing hydrogels. The decrease in the flux of glucose diffusing into the micro domains as the number of bilayers are increased accounts for the changed sensor response characteristics. Although the analytical range increases as the number of bilayers are increased, the analytical range achieved for the materials using non-cross-linked nanofilms is still not practical for in vivo sensing as it does not encompass the in vivo operational range for glucose sensors (0-400 mg/ml). All of the sensor formulations made using non-cross-linked PEMs failed to detect glucose concentration changes above 98 mg/dl. This suggests that the glucose flux into the microdomains is too high, which either overwhelms the enzyme or consumes oxygen too fast. These findings indicate that the diffusion of glucose into the sensors should be decreased further.

Table 1 shows calculated sensor figures of merit for MPACs containing non-cross-linked and cross-linked [PDADMAC/PSS]₅-[PAH/PSS]ₙ nanofilm-bounded micro domains. In each case, data from three separate MPAC hydrogels were used to calculate mean values (95% confidence).

TABLE 1

| | | | | Sensitivity/ |
|---|---|---|---|---|
| | LOD | MDGC | Range | range |
| | (mg/dl) | (mg/dl) | (mg/dl) | (% per mg/dl) |
| Uncross-linked | | | | |
| [PAH/PSS]ₙ | | | | |
| 3 | 12.0 ± 6.8 | 54.4 ± 3.2 | 40.7 ± 8.8 | 13.01 ± 4.48 |
| 5 | 15.6 ± 0.1 | 54.6 ± 4.9 | 39.8 ± 4.5 | 13.2 ± 0.60 |
| 7 | 11.5 ± 2.3 | 62.5 ± 2.1 | 51.0 ± 4.3 | 9.42 ± 1.58 |
| 9 | 14.4 ± 2.0 | 98.2 ± 7.4 | 83.8 ± 5.5 | 5.30 ± 0.46 |
| Cross-linked | | | | |
| [PAH/PSS]ₙ | | | | |
| 3 | 14.3 ± 5.0 | 65.4 ± 7.3 | 52.2 ± 11.1 | 12.54 ± 3.38 |
| 5 | 24.3 ± 4.8 | 170.8 ± 23.4 | 168.0 ± 13.4 | 2.03 ± 0.43 |
| 7 | 32.9 ± 3.7 | 296.4 ± 28.9 | 271.4 ± 23.0 | 0.86 ± 0.09 |
| 9 | 33.2 ± 9.7 | 321.2 ± 8.2 | 292.7 ± 9.5 | 0.79 ± 0.06 |

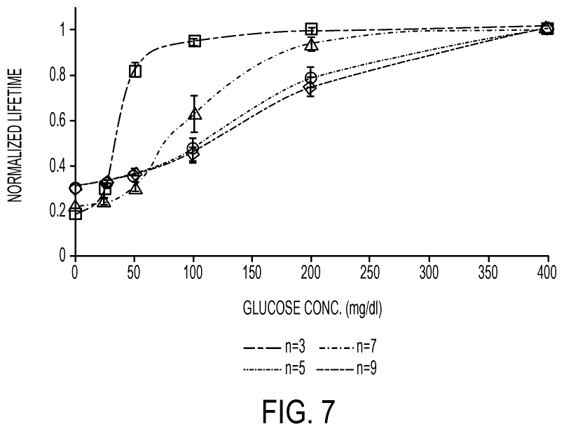

Having demonstrated that non-cross-linked nanofilm containing sensor formulations are not effective in controlling sensor dynamics considerably to engender in vivo use, the sensor response of formulations containing glutaraldehyde cross-linked microdomains were evaluated. As expected, with an increase in the number of cross-linked PAH/PSS bilayers in the nanocomposite hydrogels, the analytical range of the sensors increases and the sensitivity decreases (FIG. 7). Error bars represent 95% confidence intervals for three separate MPAC hydrogels. The analytical range increases by ≈461% while the sensitivity over the range decreases by ≈94%, as n is increased from 3 to 9. The analytical range and sensitivity of MPAC hydrogels containing cross-linked [PDADMAC/PSS]₅-[PAH/PSS]₉ was found to be ≈227% more and ≈85% less respectively than MPAC hydrogels containing non-cross-linked [PDADMAC/PSS]₅-[PAH/PSS]₉. Thus, cross-linking of PAH/PSS bilayers is crucial to significantly decrease dC/dt of glucose into the sensor and alter sensor response parameters considerably. However, as discussed previously, the rate of change of dC/dt decreases with the increase in the number of cross-linked bilayers. The decrease in the rate of change of dC/dt affects how the sensor characteristics change as number of bilayers are increased. The analytic range increases by 8% and the sensitivity over the range decreases by ≈8% when the number of cross-linked bilayers is increased from 7 to 9. This change is insignificant compared to the change in response between sensors fabricated from 3 cross-linked bilayers and 5 cross-linked bilayers. Sensor figures of merit for cross-linked microcapsule-containing hydrogels are summarized in Table 1.

It was elucidated that nano-composite hydrogels containing glucose sensing microdomains bound by a primer coating and 9 bilayers of non-cross-linked PAH/PSS were highly sensitive to glucose changes in the hypoglycemic range (<70 mg/dl) with sensor response saturation at ≈98 mg/dl. However, the in vivo use of these glucose sensing nano-composite hydrogels containing non-cross-linked nanofilm bound microdomains is impractical as the generally accepted operational range for glucose sensors is 0-400 mg/ml. By cross-linking the PAH/PSS nanofilm constructs glucose diffusion could be controlled and, more importantly, decreased sufficiently enough to effectively tune sensor characteristics. The optimized formulation of this nano-composite hydrogel containing micro-domains exhibited an operational range of 33-321 mg/dl.

The aforementioned study has shown the ability to use polyelectrolyte multilayers as the lining to a hydrogel microdomains containing encapsulated sensing chemistry. It was found possible to tune the diffusion of a model analyte (glucose) over a rather wide range by adjusting the multilayer structure by changing the composition and cross-linking. This capability is critical to engineering devices that function well in the in vivo interstitial environment, where substrate delivery may be altered from normal. The substrate permeation rate could be precisely regulated by changing the layer composition or number of spacer bilayers. On average, the cross-linked films showed an 86.27% decrease in glucose diffusion compared to non-cross-linked films, without affecting oxygen permeation. The cross-linked microcapsule sensors embedded in an MPAC hydrogel demonstrate the potential for complete control over relevant analytical range and sensitivity between 3 and 9 cross-linked layers. This provides a powerful tool to tune the dynamics of any flux-based system, which includes sensors such as the model glucose system explored here as well as controlled-release systems like medicines, fertilizers, self-healing materials, among others.

Conditional language used herein such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. While the above detailed description has shown, described, and pointed out novel features as applied to various embodiments, it will be understood that various omissions, substitutions, and changes in the form and details of the devices illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A method of fabricating a sensor, the method comprising:
forming a CaCO₃ microparticle that encapsulates at least one functional material;
coating the formed microparticle with sequential layers of polyelectrolyte film; and
coating the polyelectrolyte coated microparticle with a crosslinked alginate hydrogel.

2. The method of claim 1, wherein the CaCO₃ microparticle is formed by co-precipitating a carbonate salt and a chloride salt with the functional material.

3. The method of claim 1, wherein the functional material is a dye, nucleic acid, enzyme, inorganic nanoparticle, organic nanoparticle or drug.

4. The method of claim 1, wherein the sequential layers of the polyelectrolyte film are oppositely charged.

5. The method of claim 2, wherein the carbonate salt is Na₂CO₃.

6. The method of claim 2, wherein the chloride salt is CaCl₂.

7. The method of claim 3, wherein the dye is a phosphorescent dye.

8. The method of claim 7, wherein the phosphorescent dye is Pd-meso-tetra(4-carboxyphenyl) porphine (PdTCPP) or Pd(II) meso-tetra (sulfophenyl) tetrabenzoporphyrin (PdTSTP).

9. The method of claim 3, wherein the enzyme is glucose oxidase.

10. The method of claim 1, wherein the polyelectrolyte is Poly(diallyldimethylammonium chloride) (PDADMAC) or Poly(sodium 4-styrenesulfonate) (PSS).

11. A method of fabricating a multi-sensor hydrogel, the method comprising:
forming a CaCO₃ microparticle that encapsulates at least one functional material;
coating the formed microparticle with sequential layers of polyelectrolyte film;
coating the polyelectrolyte coated microparticle with an alginate;
mixing the alginate coated microparticle with glucone-δ-lactone;
dissolving the CaCO₃ microparticle;
releasing the functional material and Ca²⁺ ions; and
crosslinking the alginate into a hydrogel.

12. The method of claim 11, wherein the functional material is a dye, nucleic acid, enzyme, inorganic nanoparticle, organic nanoparticle or drug.

13. The method of claim 11, wherein the sequential layers of the polyelectrolyte film are oppositely charged.

14. The method of claim 12, wherein the dye is a phosphorescent dye.

15. The method of claim 14, wherein the phosphorescent dye is Pd-meso-tetra(4-carboxyphenyl) porphine (PdTCPP) or Pd(II) meso-tetra (sulfophenyl) tetrabenzoporphyrin (PdTSTP).

16. The method of claim 12, wherein the enzyme is glucose oxidase.

17. The method of claim 11, wherein the polyelectrolyte is Poly(diallyldimethylammonium chloride) (PDADMAC) or Poly(sodium 4-styrenesulfonate) (PSS).

18. A sensor formed by the method of claim 11, wherein the sensor comprises a plurality of pockets of functional material surrounded by the crosslinked alginate hydrogel.

19. The sensor of claim 18, wherein the functional material in each of the plurality of pockets is the same or different.

20. The sensor of claim 18, wherein the plurality of pockets of functional material function as sensors of glucose concentration, oxygen concentration or pH.
